(19) United States
(12) Patent Application Publication
(10) Pub. No.: US 2016/0243232 A1
(43) Pub. Date: Aug. 25, 2016

(54) PROSTATE CANCER TREATMENT

(71) Applicant: GALDERMA S.A., Cham (CH)

(72) Inventor: Andy PICKETT, Wrexham (GB)

(73) Assignee: GALDERMA S.A., Cham (CH)

(21) Appl. No.: 15/025,911

(22) PCT Filed: Sep. 29, 2014
(86) PCT No.: PCT/EP2014/070781

§ 371 Date: Mar. 30, 2016

(30) Foreign Application Priority Data
Sep. 30, 2013 (EP) 13186614.7

(51) Int. Cl. A61K 41/00; A61N 5/00; A61K 9/00; A61N 5/10; A61K 38/48; A61K 47/36

(52) U.S. Cl. CPC A61K 41/0038; A61K 38/4893; A61K 47/36; A61K 9/0019; A61K 9/0031; A61N 5/1077; A61N 5/1001; A61N 5/00; C12Y 304/24069

(57) ABSTRACT

The present invention relates to a combination of botulinum toxin and hyaluronic acid for use in the radiation treatment of cancer. The present invention also relates to a method for treating prostate cancer in a human patient, whereby one or more tumours in the prostate are shrunk, inhibited and/or dissolved and the rectum is protected from a subsequent radiation treatment step. Furthermore, the present invention relates to one or more compounds selected from botulinum toxin, hyaluronic acid and a combination of botulinum toxin and hyaluronic acid for use in the treatment of prostate cancer in a human patient by a method whereby one or more tumours in the prostate are shrunk, inhibited and/or dissolved and the rectum is protected from a subsequent radiation treatment step.

### FIELD OF THE INVENTION

The present invention relates to a combination for use in the radiation treatment of prostate cancer. The invention also relates to a method for treating prostate cancer in a human patient. Furthermore, the invention relates to one or more compounds for use in the treatment of prostate cancer in a human patient.

### BACKGROUND

Prostate cancer is a form of cancer that develops in the prostate, which is a gland of the male reproductive system. The prostate surrounds the urethra just below the urinary bladder and is situated close to the rectum.

Prostate cancer is one of the most common cancers affecting older men in developed countries and a significant cause of death for elderly men (estimated by some specialists at 3%). The presence of prostate cancer may be indicated by symptoms, physical examination, prostate-specific antigen (PSA), or biopsy. Most prostate cancers are slow growing; however, there are cases of aggressive prostate cancer. The cancer cells may metastasize (spread) from the prostate to other parts of the body, particularly the bones and lymph nodes. Prostate cancer may cause pain, difficulty in urinating, problems during sexual intercourse, or erectile dysfunction. Other symptoms can potentially develop during later stages of the disease.

Strategies for treating prostate cancer should be guided by the severity of the disease. Many low-risk tumours can be safely followed with active surveillance. Curative treatment generally involves surgery, various forms of radiation therapy, or, less commonly, cryosurgery. Hormonal therapy and chemotherapy are generally reserved for cases of advanced disease (although hormonal therapy may be given with radiation in some cases). The age and underlying health of the man, the extent of metastasis, appearance under the microscope and response of the cancer to initial treatment are important in determining the outcome of the disease. The decision whether or not to treat localized prostate cancer (a tumour that is contained within the prostate) with curative intent is a patient trade-off between the expected beneficial and harmful effects in terms of patient survival and quality of life. Studies have indicated that botulinum toxin may be useful in the treatment of prostate cancer. Chuang et al., (J. Urology, 2006, (176):2375-2382) presents a review of literature within the field of using botulinum toxin in the prostate. The document suggests that botulinum toxin may be effective in treatment of benign prostatic hyperplasia, chronic nonbacterial prostatitis and prostate cancer.

Doggweiler et al., (Prostate, 1998, 37(1): 44-50) describes a rat study where botulinum toxin is injected into prostate glands. The document concludes that there are several differences between rat and human prostate which means that the results are only valid for rat. Furthermore, the document teaches that prostate smooth muscle studies need to be performed in order to evaluate future treatment of common pathologies of the human prostate.

Karsenty et al., (Prostate, 2009, (66):1143-1150) is primarily focused on benign prostatic hyperplasia. The document discloses a study where botulinum toxin inhibits the growth of a human prostate cancer cell line, LNCaP. The document suggests future studies of the effect of botulinum toxin on human prostate cancer cells.

In summary, more studies have to be performed in order to conclude whether botulinum toxin may be an effective and a safe treatment of human prostate cancer cells.

An alternative treatment of cancer is radiation therapy, also sometimes referred to as radiotherapy. Radiation therapy is a general term used to describe several types of treatment, including the use of high-powered X-rays or placement of radioactive materials into the body. The treatment is mainly highly localized to prevent unaffected tissue damage and side effects, especially radiation toxicity.

Radiation therapy treats cancer by using high-energy radiation to destroy the cancer cells, while doing as little harm as possible to normal cells. Radiation treatment requires special consideration for the rectum, which is particularly sensitive to radiation. Rectal radiation injury can for example result in diarrhoea, and/or rectal urgency. Hence, care must be taken to ensure that the rectum receives a radiation dose that is well tolerated, preferably no dose at all, and to diminish the probability of treatment-related side-effects. Wilder et al., (Int. J. Radiation Oncology Biol. Phys., 2010, (77) 3:824-830) discusses that cross-linked hyaluronan gel can reduce the acute rectal toxicity of radiotherapy for prostate cancer.

The international patent application WO 2006/094539 discloses various methods and compositions for the treatment of cancer. For example, radiotherapy can be combined with chemotherapy. Different sensitizers may be used in combination with radiotherapy and/or chemotherapy. For example, tumours can be sensitised to cytotoxic therapies when they are pre-treated with a botulinum toxin. The combination of radiotherapy and administration of botulinum toxin to cancerous cells appears more effective than only using radiotherapy. The administration of botulinum toxin sensitises cancerous cells or tumour to subsequent treatment with radio- or chemotherapy. According to the study, the botulinum toxin is not an anti-cancer agent in itself, but merely a sensitiser.

### DISCLOSURE OF THE INVENTION

It is an object of the present disclosure to alleviate at least some of the problems associated with the prior art techniques.

It is an object of the present disclosure to provide a new method for treatment of prostate cancer in a human patient.

In particular, it is an object of the disclosure to provide a new method for use in the treatment of prostate cancer in a human patient, whereby one or more tumours in the prostate are shrunk, inhibited and/or dissolved.

It is another object of the disclosure to provide a new method for use in the treatment of prostate cancer, whereby the rectum of the patient is at least partially if not fully protected from radiation during at least one subsequent treatment step.

The above mentioned objects, as well as other objects that will be apparent to a person skilled in the art when presented with the present disclosure, are each addressed by at least one of the different aspects of the present invention.

In a first aspect thereof, the present invention provides a combination of botulinum toxin and hyaluronic acid for use in the radiation treatment of prostate cancer.

The invention is generally based on the insight that the combined use of these substances in a radiation therapy regime against prostate cancer act synergistically with the radiation effects from the radiation. The effect of the cooperative function of botulinum toxin and hyaluronic acid in combination with radiotherapy is unexpected.

The present invention makes use of both a composition comprising botulinum toxin and a composition comprising hyaluronic acid in the treatment of prostate cancer. More specifically, the invention is based on the inventive realization that botulinum toxin may be used in order to shrink, inhibit and/or dissolve one or more cancer tumours in the prostate and as a prior, simultaneous or subsequent step, hyaluronic acid is introduced as a space-forming material between the prostate and the rectal wall in order to protect the rectum during radiation therapy. The result is an improved treatment of prostate cancer which improves the targeting of the radiation therapy and minimizes the toxicity experience by the rectal wall during radiation treatment.

In the context of the present disclosure, “Botulinum toxin” means a botulinum neurotoxin produced by Clostridium botulinum or Clostridium baratii, as well as a botulinum toxin (or the light chain or the heavy chain thereof which are subsequently combined together) made by genetic recombinant techniques using a non-Clostridial species. The phrase “botulinum toxin”, as used herein, encompasses the botulinum toxin serotypes A, B, C, D, E, F and G and their subtypes. Botulinum toxin, as used herein, also encompasses both a botulinum toxin complex (i.e. the complexes of approximate molecular weight 300, 600 and 900 kDa or other complexes as produced naturally by the bacteria) as well as the purified botulinum toxin (approximately 150 kDa molecular weight). “Purified botulinum toxin” is defined as a botulinum neurotoxin that is isolated, separated or substantially isolated or separated, from other proteins, including proteins that form a botulinum toxin complex. A purified botulinum toxin may be greater than 90% pure, preferably greater than 95% pure and most preferably greater than 99% pure. Botulinum toxin, as used herein, also encompass modified botulinum toxin. Modified botulinum toxin means a botulinum toxin that has at least one of its amino acids deleted, modified, or replaced, as compared to native botulinum toxin. The modified botulinum toxin retains at least one biological activity of the native botulinum toxin. Furthermore, the modified botulinum toxin can be recombinantly produced botulinum toxin or a derivative or fragment of a recombinantly made botulinum toxin. In a preferred embodiment according to the present invention, the botulinum toxin is botulinum toxin type A subtype A1.

Throughout the present disclosure, the term “hyaluronic acid” refers to a compound constituted of series of glucuronic acid and of N-acetylglucosamine. Hyaluronic acid can be in the form of a pharmaceutically acceptable salt or a derivative thereof, particularly a sodium or potassium salt. Hyaluronic acid may be used in various forms: a salt, a derivative such as an ester or an amide, in linear or cross-linked form.

In a preferred embodiment, the hyaluronic acid is cross-linked. “Cross-linking” refers to a process in which the individual chains of hyaluronic acid are chemically bound (or “cross-linked”) together into a soft solid, or “gel.” The strength or firmness of the gel depends on the degree of cross-linking of the individual hyaluronic acid chains. The body metabolizes cross-linked hyaluronic acid more slowly than natural, unlinked hyaluronic acid, resulting in a longer duration of effect when used for aesthetic or therapeutic indications. For example, according to the present invention the hyaluronic acid can be in the form of a gel. In another example, the hyaluronic acid is in the form of gel particles. A person skilled in the art may determine the size of the particles. A preferable way of producing a cross-linked hyaluronic acid according to the invention is according to WO 97/04012.

In the context of the present disclosure, a “combination of botulinum toxin and hyaluronic acid” means that both botulinum toxin and hyaluronic acid are utilized when treating prostate cancer. The term “combination” includes a single composition comprising each of the botulinum toxin and hyaluronic acid, but also includes a kit comprising at least two different compositions, each comprising a part of either the botulinum toxin or the hyaluronic acid. The term “combination” also includes two different compositions, each comprising a part of either the botulinum toxin or the hyaluronic acid. According to the present disclosure, hyaluronic acid may be administered prior to or simultaneously as the botulinum toxin or as a subsequent step in a treatment of prostate cancer. In another example, botulinum toxin may be administered prior to or simultaneously as the hyaluronic acid or as a subsequent step in the treatment of prostate cancer.

“Prostate cancer” means cancer in the prostate which is a part of the male reproductive system.

In the context of the present disclosure, “treating” refers to alleviating (or even eliminating) at least one symptom of prostate cancer, either temporarily or permanently. A temporarily treated person having prostate cancer may be subjected to subsequent treatment of prostate cancer. In another example, a temporarily treated person suffering from prostate cancer may be subjected to reoccurrence of prostate cancer, i.e. tumour growth. The development of prostate cancer in a patient can be evaluated by determining prostate specific antigen (PSA) levels. The level of PSA increases with progression of prostate cancer.

Prostate cancer is a common type of cancer affecting men, often elderly men. The prostate is a gland which surrounds the urethra just below the urinary bladder and is situated close to the rectum. Prostate cancer is commonly treated by using radiation therapy. While prostate radiation therapy has advantages relative to other treatments, one issue is radiation injury to the rectum, since the rectum is positioned immediately posterior to the prostate.

Without wishing to be bound to any specific theory, botulinum toxin is thought to affect nerve terminals in the prostate and the release of neurotransmitters including acetylcholine, sensory neuropeptides, and noradrenalin. These effects may alter neural control within the prostate. Botulinum toxin is also thought to have a role in the management of prostate cancer, possibly by inhibiting inflammation and the down regulation of COX-2 expression.

Botulinum toxin may be used when treating prostate cancer in order to shrink one or more tumours situated in the prostate (Doggweiler R. et al, Prostate, 1998:37(1):44-50; Vezdrevanis K, Urol J, 2011; 8, 239-41). Other studies indicate that botulinum toxin inhibits the growth of tumour cells in the prostate (Karsenty G et al, Prostate. 2009 Aug. 1; 69(11):1143-50). The result may be smaller or even dissolved tumour(s).

Hyaluronic acid (also called hyaluronate or abbreviated HA) is an anionic, non-sulfated glycosaminoglycan distributed widely through connective, epithelial and neural tissues. Hyaluronic acid is one component of the extracellular matrix and contributes to cell proliferation and migration. Treatment containing hyaluronic acid is ideally suited for therapeutic and aesthetic applications because of the critical role that the material has in biological processes and its biocompatibility through the dermis.

There are several advantages of using a combination of botulinum toxin and hyaluronic acid in the treatment of prostate cancer.

One advantage of using botulinum toxin in prostate cancer treatment is that botulinum toxin may decrease the size of one or more tumours in the prostate. Another advantage is that botulinum toxin may inhibit tumour cell growth in the prostate. Since tumour(s) is/are shrunk and/or growth of tumour(s) is inhibited, this may facilitate subsequent treatment steps, for example in the form of radiation, since then smaller tumour(s) need to be radiated. A more focused treatment with radiation is therefore possible. In some examples, a smaller radiation dose may be needed during treatment. In other examples, fewer radiation occurrences may be needed during treatment. Shrinking the prostate cancer tumour(s) reduces the size of the target(s) to be irradiated, which provides additional safety margin(s) in subsequent radiation treatment. It is contemplated that this is advantageous to further reduce the side effects associated with radiation therapy of prostate cancer.

According to the present invention, botulinum toxin is utilized as an anti-cancer agent. The botulinum toxin is active in the treatment of cancer, such as prostate cancer. After administration of botulinum toxin, the tumour will decrease in size or at least its growth will be inhibited or delayed. Hence, the administration of botulinum toxin to cancer cells is an active treatment of the cancer.

According to the present invention, administration of botulinum toxin is combined with radiotherapy as an additional treatment step. To dispel any doubts, botulinum toxin is not used in order to sensitize a cancer tumour for additional treatment, such as chemotherapy. Instead, botulinum toxin is an active substance on its own merits.

Furthermore, the botulinum toxin molecule is rather large and has a limited diffusion ability and thus, botulinum toxin can be prohibited from reaching outside the area where it is administered, e.g. to the prostate.

Hyaluronic acid may be used in order to protect the rectum of the patient to be treated by radiation therapy. Hyaluronic acid may act as a physical spacer and can be administered to the area between the prostate and the rectum. Time, distance and shielding affect the radiation dose that is delivered. For example, the greater distance from the radiation, the smaller dose is delivered. The hyaluronic acid spacer increases the distance between the prostate (high dose radiation area) and surrounding tissue, such as the rectum. Due to the physical spacer, surrounding tissue, such as the rectum, is exposed to less radiation compared to the condition without the presence of a physical spacer. Hence, one advantage of using a physical spacer of hyaluronic acid during treatment of prostate cancer is that unintended rectal irradiation may be substantially reduced or even eliminated.

Another advantage of using a physical spacer of hyaluronic acid is that a higher dose of cancer radiation per treatment could be allowed. Potentially, this would lead to improved patient survival. Also, this may result in fewer visits to hospital for treatment resulting in increased patient convenience and decreased healthcare costs.

Furthermore, hyaluronic acid is a biodegradable material. The material does not require manipulation, repositioning, or removal from the body. Hyaluronic acid may maintain its natural properties during treatment and may remain in the same location until after a treatment course has concluded.

As a result, the method for treating prostate cancer in a human patient, as described herein, results in a more convenient cancer treatment for the patient. The method may result in fewer visits to the hospital and lower risk for rectal injury. This may also lead to decreased healthcare costs.

In summary, a treatment course utilizing both botulinum toxin and hyaluronic acid results in an improved treatment of prostate cancer. The proposed combination acts jointly and brings additional safety benefits for patients undergoing prostate irradiation treatment, to minimise toxic side effects from the radiation and provide a greater standard of care.

In a second aspect thereof, the present invention provides a method for treating prostate cancer in a human patient, comprising the steps of:
a) administering a first composition comprising a therapeutically active amount of a botulinum toxin to the prostate of said patient,
b) allowing the botulinum toxin to act on one or more tumours in the prostate, whereby one or more tumours in the prostate are shrunk, inhibited and/or dissolved,
c) administering a second composition comprising hyaluronic acid to a location between the prostate and rectum of said patient to protect the rectum from a subsequent radiation treatment step d,
d) exposing the prostate of said patient to radiation therapy, whereby one or more tumours in the prostate are shrunk, inhibited and/or dissolved.

The following description of embodiments, features and characteristics is equally applicable to all aspects of the invention as described herein.

Throughout the present disclosure, “patient” means a human receiving medical care.

In the context of the present disclosure, “administration”, “administering” or “to administer” refer to the step of giving (i.e. administering) a composition to a subject. The compositions disclosed herein can be administered by e.g. intramuscular administration (into a muscle), transrectal administration (via rectum), intravesicular infusion into the urinary bladder, intracavernous administration (injection into the base of the penis), intraprostatic administration (injection into the prostate), transperineal administration (via skin to the prostate), infraperitoneal/subperitoneal administration (injection e.g. into the rectum), intraperitoneal administration (infusion or injection into the peritoneum) and/or transurethral administration (via urethra).

The compositions of the present invention can be administered by any suitable route. For example, the compositions may be administered with an injection. In other examples, the compositions are administrated to the area where the composition is to act. One example can be to the prostate with transperineal injection, or in the area between the prostate and the rectum with an injection.

The compositions of the present invention can be administered by any suitable means. For example, injection can be performed using a syringe and a needle.

The administration of compositions disclosed herein may be guided by using transrectal ultrasound where an ultrasound probe is placed in the rectum during administration.

The administration of compositions disclosed herein may also be administered by transurethral routes using cystoscopy for guidance.

The compositions disclosed herein may be administered by trained personnel using aseptic techniques.

The botulinum toxin can be formulated in any pharmaceutically applicable formulation such as liquid, powder, cream, emulsion, suspension, solution, etc.

A therapeutically effective amount of the botulinum toxin is the dosage sufficient to achieve the desired treatment outcome. For example, the amount sufficient to inhibit neuronal activity or to prevent neurotransmitter release for at least one week, more preferably at least one month, most preferably for approximately 6 months or longer. Preferably, the dose administered to the patient may be any dose less than a toxic dose. The volume of the composition and the concentration of the botulinum toxin and the number of administrations may depend upon the size of the patient, the cancer development, size of prostate, number and size of tumours in the prostate, potency of the botulinum toxin, and other factors.

Dosing can be single dosage or cumulative (serial) dosing and can be readily determined by one skilled in the art. Botulinum toxin may be administered at least once. For example, the number of doses depends on the desired effect and potency between the serotype of botulinum toxin utilized, as well as the amount of total toxin and dilution of toxin utilized. The botulinum toxin can be delivered serially (i.e. once per month, once every second month) so that the therapeutic effect can be optimized. Divided doses over a span of time, such as a period of days or weeks or months, may depend on the length of effect for a given botulinum toxin preparation.

The dosing of botulinum toxin will depend on the preparation to be used since the potency units of each preparation are specific to that preparation alone. A typical range of a single dose might be from 50-1000 potency units and this dose may be repeated on multiple occasions to provide the desired effect on the prostate tumours.

Typically, no less than about 1 unit and no more than about 2500 units of a botulinum toxin type A (such as Botox®) can be administered per injection site, e.g. during one treatment session. For a botulinum toxin type A such as DYSPORT® no less than about 2 units and no more than about 4000 units of the botulinum toxin type A can be administered per injection site, e.g. during one treatment session. The volume of the botulinum toxin to be administered depends e.g. on the concentration of the botulinum toxin, the serotype of botulinum toxin selected and the brand of the botulinum toxin selected. In an embodiment, between about 1 unit and about 1500 units of botulinum toxin type A per patient (e.g. a 70 kg man) per treatment is administered. In another embodiment, between about 50 units and about 500 units of botulinum toxin type A per patient (e.g. a 70 kg man) per treatment is administered. In a preferred embodiment, between about 100 units and about 200 units of botulinum toxin type A per patient (e.g. a 70 kg man) per treatment is administered.

Before administration, the botulinum toxin may be reconstituted with e.g. saline 0.9%.

The dosage schedule can be readily determined by one skilled in the art based on for example patient size, and will depend on many factors, including the botulinum toxin selected, the degree of cancer development, and other variables.

With “target” it is meant the location/area, tissue or gland of the patient's anatomy in which the desired effect of the administered composition is exerted. The target of the composition comprising a botulinum toxin is the prostate in the human body. Preferably, the target is one or more tumours in the prostate.

The botulinum toxin is allowed to act on one or more tumours in the prostate, whereby one or more tumours in the prostate are shrunk, inhibited and/or dissolved. For example, botulinum toxin is allowed to act on one or more tumours during at least 1 hour, such as during at least 2 hours, such as during at least 5 hours, such as during at least 1 day, such as during at least 1 week prior to a subsequent treatment step for example in the form of radiation treatment. It is believed that the reason for the advantage of using a botulinum toxin in the treatment of prostate cancer is that tumour cell growth is inhibited by the botulinum toxin, although the disclosure of the present invention is not intended to be bound by any specific theory. The composition comprising a botulinum toxin may also destroy tumour cells and the result is shrunk or even dissolved tumours.

The composition comprising hyaluronic acid can be formulated in any pharmaceutically applicable formulation such as liquid, powder, cream, emulsion, suspension, solution, gel, particles, etc.

A desired amount of hyaluronic acid to be administered to the patient is a dosage sufficient to achieve the desired treatment outcome. The volume of the composition and the concentration of the hyaluronic acid may depend upon the size of the patient, the cancer development, size of the prostate, and other factors. For example, a volume of 1-20 ml can be administered, such as 2-15 ml, such as 3-10 ml.

The dosage schedule can be readily determined by one skilled in the art based on for example patient size, and will depend on many factors, including the degree of prostate cancer development, and other variables. Hyaluronic acid may be administered at least once. The hyaluronic acid may be administered as a single dose or as serial doses. The number of doses depends on the desired effect. One administration of hyaluronic acid may be satisfactory for several occurrences of radiation treatment. A large volume of hyaluronic can create a large physical space between the prostate and the rectum compared with a smaller volume of hyaluronic acid. In another example, serial doses of a small quantity of hyaluronic acid administered with a small time interval may build up a large physical spacer of hyaluronic acid between prostate and rectum. In another example, new administration of hyaluronic acid may be needed because earlier administered hyaluronic acid is at least partly biodegraded.

The target of the composition comprising hyaluronic acid is an area between the prostate and the rectum in the human body.

The administration of hyaluronic acid into the area between the prostate and the rectum, or rectal walls, protects, at least to a part, the rectum during simultaneous or subsequent radiation treatment step(s). For example a subsequent treatment step can be radiation therapy. The hyaluronic acid acts as a physical spacer between the prostate and the rectum and increases the distance between the prostate and the rectum, resulting in less unintended radiation reaching the rectum when the prostate is exposed to radiation. “Protected at least to a part” and “at least partially protected” means that although the rectum is protected compared to the situation without a physical spacer, the rectum may nevertheless be subjected to some radiation.

The radiation therapy may be performed simultaneously with, immediately after or after administration of hyaluronic acid. In some examples, radiation treatment can be performed afterwards, such as after 5 minutes, such as after 1 hour, such as after 2 hours, such as after 1 day, such as after 1 week, such as after more than 1 week, after administration of hyaluronic acid.

The size of the physical spacer of hyaluronic acid may determine the dose of radiation to which the rectum is exposed. For example, the physical spacer of hyaluronic may increase the distance between prostate and rectum with more than 1 mm, such as more than 2 mm, such as more than 5 mm, such as more than 1 cm, and less than 10 cm, or such as less than 2 cm.

The physical spacer of hyaluronic acid is biocompatible and introduction of hyaluronic acid into the body will not induce any severe or permanent side effects.

The physical spacer of hyaluronic acid is biodegradable. The physical spacer of hyaluronic acid need preferably not be removed from the body after radiation treatment has ended. The time for its biodegradability may be determined by the time required to complete a course of radiation therapy.

During treatment of prostate cancer, the prostate of the patient is exposed to radiation therapy. The radiation therapy can be performed with a single occurrence or with several occurrences. In one example, the radiation therapy is performed at one time but with intervals, such as with pulses. The radiation therapy schedule can be readily determined by one skilled in the art based on for example patient size, and will depend on many factors, including the presence of physical spacer of hyaluronic acid selected, the degree of cancer development, and other variables.

Radiation treatments are used in a wide range of settings. The circumstances include primary treatment of localized prostate cancer, secondary treatment for cancer recurring within the region of the prostate and for relief of pain and other symptoms related to prostate cancer that has spread to other parts of the body.

Although examples of routes of administration and dosages are provided herein, the appropriate route of administration and dosage are generally determined on a case by case basis by attending physician. The route and dosage can be selected based upon criteria such as the characteristics of the composition selected as well as the development of the prostate cancer.

As described herein, step a) is typically performed before step c), i.e. botulinum toxin is typically administered before hyaluronic acid. In other examples, step c) may however be performed before or simultaneously with step a), i.e. hyaluronic acid may be administered before or at the same time as administration of botulinum toxin. Since hyaluronic acid is a biocompatible material, no or few side-effects are known and hence, introduction into the body will not lead to any inconvenience to the patient. However, care has to be taken to the fact that hyaluronic acid is biodegradable. If hyaluronic acid is administered and the course of treatment of botulinum toxin administration, and subsequent steps of radiation treatment, occurs for a long period of time, additional administration of hyaluronic acid may be needed. In all cases, administration of hyaluronic acid is performed before a treatment step of radiation.

As set out above, step a) is performed before step b). Furthermore, step c) is performed before step d). In the light of the present disclosure, step a) and b) is preferably performed before step d). Botulinum toxin is preferably administered to the prostate and allowed to act on one or more tumours before a subsequent treatment step, e.g. in the form of radiation therapy.

In a preferred embodiment of the present invention, the first composition comprising a botulinum toxin is administered by injection. Such injection can be administered by using for example a syringe and a needle. The injection may be with a single dose or divided into several dosages. For example, administration of the botulinum toxin can be achieved by less than 20 injections into or in the vicinity of the prostate, more preferably less than 10 injections into or in the vicinity of the prostate, and most preferably by performing between 1 and 5 injections into or in the vicinity of the prostate.

In an embodiment of the present invention, the first composition comprising a botulinum toxin is administered by transperineal injection.“Transperineal injection” refers to an injection where a (thin) needle is inserted through the skin between the scrotum and rectum and into the prostate. In another example, transrectal injection is used. In another embodiment of the present invention, the first composition comprising a botulinum toxin is administered by transrectal injection. “Transrectal injection” means an administration route where an injection is performed using a (thin) needle which is inserted via the rectum into the prostate. However, a potential drawback with transrectal injection may be bacterial contamination of the injected prostate. In yet another embodiment of the present invention, the first composition comprising a botulinum toxin is administered by transurethral injection. “Transurethral injection” refers to an administration route where injection is performed into the prostate via the urethra.

Ultrasound and magnetic resonance imaging (MRI) are two commonly used imaging methods for prostate cancer. For example, urologists use transrectal ultrasound during prostate biopsy. MRI has superior soft tissue resolution compared to ultrasound. MRI uses magnetic fields to locate and characterize prostate cancer. MRI can be used alone or in combination with ultrasound guidance. Hence, the assistance of e.g. ultrasound and/or MRI may guide the person performing an injection of a composition according to the present disclosure to locate a target to inject the composition into. In one embodiment, the botulinum toxin is injected into the prostate with guidance of ultrasound. In another embodiment, the botulinum toxin is injected into the prostate with guide of MRI.

In an example, the composition comprising a botulinum toxin may comprise a contrast medium, or the like, to be able to use an imaging technique for guidance. “Contrast medium” (or contrast agent) means a substance used to enhance the contrast of structures or fluids within the body in medical imaging. In another example, a contrast medium, or the like, is administered to the patient prior to administration of a composition comprising a botulinum toxin. Examples of contrast mediums are radiocontrast medium (contrast medium for enhancing x-ray-based imaging methods), such as iodine and barium, and MRI contrast agent, such as gadolinium.

In a preferred embodiment of the present invention, the second composition comprising hyaluronic acid is administered by injection. Such injection can be administered by using for example a syringe and a needle. The injection may be with a single dose or divided into several dosages. For example, administration of hyaluronic acid can be achieved by less than 20 injections into the area between the prostate and the rectum, more preferably less than 10 injections into the area between the prostate and the rectum and most preferably by performing between 1 and 5 injections into the area between the prostate and the rectum.

In an embodiment of the present invention, the second composition is administered by using transperineal injection. In another embodiment, transrectal injection is used.

It is preferred that the composition comprising hyaluronic acid is administered into Denovillier's space. “Denovillier's space” is a region located between the rectum and prostate. It consists of a single fibromuscular structure covering the posterior part of the prostate. The space separates the prostate from the anterior rectal wall. It may be important that the composition comprising hyaluronic acid is administered on the posterior side of the Denovillier's space and anterior to the anterior rectal wall in order to minimize the risk of pushing cancer cells away from the high dose radiation field.

Hydrodissection is a technique used to separate tissue planes through the use of fluid. For example, a saline solution can be injected into the Denovillier's space prior to administration of a composition comprising hyaluronic acid. In an example, a saline injection opens the space between the prostate and the rectal wall, which allows needle advancement to the desired location without rectal wall injury. A composition comprising hyaluronic acid is then injected into the Denovillier's space using a transperineal approach under real-time transrectal ultrasonography guidance.

In an embodiment, the first composition comprising a botulinum toxin is administered to the surface area of the prostate. The composition comprising botulinum toxin may diffuse throughout the prostate. The botulinum toxin may reach and penetrate one or more tumours. However, the botulinum toxin molecule/complex may be large and the diffusion rate may be low.

In another embodiment, the first composition comprising a botulinum toxin is administered directly to one or more tumours in the prostate, i.e. into or in the vicinity of the tumours.

Botulinum toxin can be divided into seven serologically distinct toxin types which are designated A through G. Each serotype has a series of subtypes. The different serotypes and subtypes of botulinum toxin vary in animal species that they affect and in the severity and duration of the paralysis they evoke in each species.

In a preferred embodiment in accordance with the present invention, the botulinum toxin is botulinum toxin type A, e.g. in a protein complex or purified form. In a more preferred embodiment, the botulinum toxin is a purified botulinum toxin type A. In one embodiment, the botulinum toxin is a purified botulinum toxin subtype A1, e.g. in a protein complex or purified form. In a more preferred embodiment, the botulinum toxin is a purified botulinum toxin type A1.

For examples, the botulinum toxin type A is Botox, Dysport, Azzalure, which are trade names. They are often used for various aesthetic and medical procedures.

In a preferred embodiment of the present invention, the hyaluronic acid is cross-linked. In an example, the hyaluronic acid is in the form of a gel. In another example, the hyaluronic acid is in the form of gel particles, typically having a size in the range of 0.05-5 mm, such as 0.1-1.5 mm. A person skilled in the art may determine a suitable size of the particles.

In a preferred embodiment of the present invention, the radiation therapy is selected from external beam radiation therapy (EBRT), proton therapy and brachytherapy.

An example of EBRT is intensity modulated radiotherapy. A patient to be treated sits or lies on a couch and an external source of radiation is pointed at a particular part of the body. Another example is hypofractionated irradiation therapy. In this type of radiation treatment, the total dose of radiation is divided into large doses and treatments are given less than once a day. Yet another example is a specialized type of external beam radiation therapy called stereotactic radiation, which uses focused radiation beams targeting a well-defined tumour, relying on detailed imaging. EBRT may case radiation-induced side effects for prostate cancer, especially on the rectum.

Proton therapy is a type of external beam radiotherapy using ionizing radiation. It is a type of particle therapy which uses a beam of protons to irradiate diseased tissue, most often used in the treatment of cancer.

Brachytherapy is also known as internal radiotherapy and is a form of radiotherapy where a radiation source is placed inside or next to the area requiring treatment. Brachytherapy involves the precise placement of short-range radiation-sources (radioisotopes) directly at the site of the cancerous tumour. These are enclosed in a protective capsule or wire which allows the ionizing radiation to escape to treat and kill surrounding tissue, but prevents the charge of radioisotope from moving or dissolving in body fluids. The capsule may be removed later, or (with some radioisotopes) it may be allowed to remain in place. A key feature of brachytherapy is that the irradiation only affects a localized area around the radiation sources. Exposure to radiation of healthy tissues further away from the sources is therefore reduced. In addition, if the patient moves or if there is any movement of the tumour within the body during treatment, the radiation sources retain their correct position in relation to the tumour.

For example, ultrasound-guided transperineal interstitial permanent prostate brachytherapy with 1-125 (Iodine 125) or Pd 103 (Palladium 103) radioactive seeds is a form of radiation therapy in which radioactive sources, or “seeds”, are permanently inserted into the prostate. The principal advantage of this technique is that the seeds can deliver a substantially higher radiation dose to the prostate and surrounding tissue compared with external beam irradiation. Because of the low energy of 1-125 and Pd 103 isotopes, the dose falls off quickly with distance and, therefore, the seeds deliver low doses to the adjacent rectum and bladder.

Even with this type of radiation treatment, the rectum may be exposed to radiation and a physical spacer of hyaluronic acid would be suitable.

In a third aspect thereof, the present invention provides one or more compounds selected from botulinum toxin, hyaluronic acid and a combination for botulinum toxin and hyaluronic acid for use in the treatment of prostate cancer in a human patient by a method comprising the steps of:
a) administering a first composition comprising a therapeutically active amount of a botulinum toxin to the prostate of said patient,
b) allowing the botulinum toxin to act on one or more tumours in the prostate, whereby one or more tumours in the prostate are shrunk, inhibited and/or dissolved,
c) administering a second composition comprising hyaluronic acid to a location between the prostate and rectum of said patient to protect the rectum from a subsequent radiation treatment step d,
d) exposing the prostate of said patient to radiation therapy, whereby one or more tumours in the prostate are shrunk, inhibited and/or dissolved.

The combination in the third aspect may comprise any feature mentioned herein in relation to the combination of the first and second aspect of the invention. The method steps in the third aspect may comprise any feature mentioned herein in relation to the method steps of the second aspect of the invention.

Botulinum toxin can be used in the treatment of prostate cancer in a human patient by a method comprising the steps a-d described herein. Hyaluronic acid can be used in the treatment of prostate cancer in a human patient by a method comprising the steps a-d described herein. Furthermore, a combination of botulinum toxin and hyaluronic acid may be used in the treatment of prostate cancer in a human patient by a method comprising the steps a-d described herein. For example, a composition comprising botulinum toxin, a composition comprising hyaluronic acid or a kit comprising both botulinum toxin and hyaluronic acid may be used. The amount of botulinum toxin and the amount of hyaluronic acid may be determined by a person skilled in the art.

1-8. (canceled)

9. A method for treating prostate cancer in a human patient, comprising:
a) administering a first composition comprising a therapeutically active amount of a botulinum toxin to a prostate of said patient,
b) allowing the botulinum toxin to act on one or more tumours in the prostate, whereby one or more tumours in the prostate are shrunk, inhibited and/or dissolved,
c) administering a second composition comprising hyaluronic acid to a location between the prostate and rectum of said patient to protect the rectum from a subsequent radiation treatment step d,
d) exposing the prostate of said patient to radiation therapy, whereby one or more tumours in the prostate are shrunk, inhibited and/or dissolved.

10. A method according to claim 9, wherein said administering of said first composition is accomplished by injection.

11. A method according to claim 9, wherein said administering of said second composition is accomplished by injection.

12. A method according to claim 9, wherein the botulinum toxin is Botulinum toxin type A.

13. A method according to claim 12, wherein the Botulinum toxin type A is selected from the subtypes A1, A2, A3, A4 and A5.

14. A method according to claim 9, wherein the hyaluronic acid is cross-linked.

15. A method according to claim 9, wherein said radiation therapy is selected from external beam radiation therapy (EBRT), proton therapy and brachytherapy.

16. A method for treating prostate cancer in a human patient, comprising administering a combination of botulinum toxin and hyaluronic acid to a prostrate of said patient.

17. A method according to claim 16, wherein the combination of botulinum toxin and hyaluronic acid is a kit comprising a first composition comprising a therapeutically active amount of the botulinum toxin and a second composition comprising hyaluronic acid, wherein the first composition and second composition are different.

18. A method according to claim 17, wherein the method comprises administering the first composition to a prostate of said patient and administering the second composition between the prostate and rectum of said patient.

19. A kit for use in radiation treatment of prostate cancer, the kit comprising a first composition comprising a botulinum toxin and a second composition comprising hyaluronic acid, wherein the first composition and second composition are different, wherein the first composition and the second composition are configured for use in the radiation treatment of prostate cancer in a human patient.
